(19) United States
(12) Patent Application Publication
(10) Pub. No.: US 2024/0374408 A1
(43) Pub. Date: Nov. 14, 2024

(54) ASSISTIVE ADAPTER DEVICES AND MOUNTING SYSTEMS FOR INDIVIDUALS WITH LIMITED HAND FUNCTIONS

(71) Applicant: REACHABLE TECHNOLOGY LLC, Cambridge, MA (US)

(72) Inventors: William M. MUTER, Cambridge, MA (US); Matthew GUY, Lowell, MA (US); Santiago HIRSCHMANN, Dartmouth, MA (US)

(21) Appl. No.: 18/658,691

(22) Filed: May 8, 2024

Related U.S. Application Data
(60) Provisional application No. 63/465,111, filed on May 9, 2023.
(60) Provisional application No. 63/530,772, filed on Aug. 4, 2023.
(60) Provisional application No. 63/563,744, filed on Mar. 11, 2024.

(51) Int. Cl. A61F 4/00

(52) U.S. Cl. CPC A61F 4/00

(57) ABSTRACT

An accessibility system for assisting with access and use of any of a plurality of tools is disclosed. The accessibility system includes a base-mount, an adaptor and a universal connector. The base-mount is for fixed attachment within an environment is provided. The base-mount includes at least one vertically extended portion. The adaptor is for attachment to an extremity of a user without the need to grip the adapter. The universal connector includes a tool attached thereto, and a base-mount cavity for receiving the vertically extending portion of the base-mount. The adaptor and the universal connector each include one of a latch projection and a latch cavity that receives the latch projection.

### PRIORITY

The present invention claims priority to U.S. Provisional Application No. 63/465,111, filed May 9, 2023, U.S. Provisional Application No. 63/530,772, filed Aug. 4, 2023, and U.S. Provisional Application No. 63/563,744, filed Mar. 11, 2024, the disclosures of each are hereby incorporated by reference in their respective entirety.

### BACKGROUND

The invention generally relates to adaptive devices for assisting persons with limited movement and/or dexterity, and relates in particular to adaptive devices the providing such persons with the ability to use a wide variety of objects.

Certain adaptive devices (such as universal cuffs) allow individuals with limited hand function the ability to use everyday objects such as utensils, writing objects and grooming products. While the use of such devices (when a tool is engaged with the universal cuff) increases the independence of the user, the insertion and replacement of tools (e.g., changing from a fork to a spoon) generally requires a dependence of another individual due to the difficulty of tool replacement.

U.S. Pat. No. 11,090,178, discloses an adaptive device for a hand that includes a connection member with spring loaded balls on an outer surface thereof that received within recesses of a retention member. A release lever is disclosed to be used by urging the release lever against compressed springs to release the spring-loaded balls from the recesses.

There remains a need for an adaptive system for permitting access by persons of limited movement and/or dexterity in using a wide variety of tools without requiring another person to engage or disengage each too.

### SUMMARY

In accordance with an aspect, the invention provides an accessibility system for assisting with access and use of any of a plurality of tools. The accessibility system includes a base-mount, an adaptor, and a universal connector. The base-mount is for fixed attachment within an environment, and includes at least one vertically extended portion. The adapter is for attachment to an extremity of a user without the need to grip the adapter. The universal connector includes a tool attached thereto, and a base-mount cavity for receiving the vertically extending portion of the base-mount. The adaptor and the universal connector each include one of a latch projection and a latch cavity that receives the latch projection.

In accordance with another aspect, the invention provides an accessibility system for assisting with access and use of any of a plurality of tools. The accessibility system includes a base-mount, an adaptor and a universal connector. The base-mount is for fixed attachment within an environment, and the base-mount includes a plurality of vertically extended portions. The adapter is for attachment to an extremity of a user without the need to grip the adapter. The universal connector includes a tool attached thereto. The adaptor and the universal connector include a latch projection and a latch cavity that receives the latch projection. The base-mount cavity is for receiving a vertically extending portion of the base-mount of the plurality of vertically extending portions.

In accordance with a further aspect, the invention provides a method of assisting a user with access to and use of any of a plurality of tools. The method includes: moving an adaptor that is attached to an extremity of a user toward a universal connector, the universal connector including a tool attached thereto and being mounted on a vertically extended portion of a base-mount; contacting the adaptor with the universal connector to attach the universal connector to the adaptor; and elevating the universal connector with the adaptor upward off of the vertically extended portion of the base-mount.

### BRIEF DESCRIPTION OF THE DRAWINGS

The following description will be further understood with reference to the accompanying drawings in which:

FIGS. 1A-1D show illustrative diagrammatic views of a singular base-mount used in an embodiment of the invention;

FIGS. 2A-2C show illustrative diagrammatic views of a tool extender used in an embodiment of the invention;

FIGS. 3A-3F show illustrative diagrammatic views of a universal tool mount used in an embodiment of the invention;

FIGS. 4A-4F show illustrative diagrammatic views of an adapter element of the present invention;

FIGS. 5A-5E show illustrative diagrammatic views of an adapter extender of the present invention;

FIGS. 6A-6C show illustrative diagrammatic views of the adapter strap used in an embodiment of the present invention;

FIG. 7 shows an illustrative diagrammatic view of a fork orientation;

FIG. 8 shows an illustrative diagrammatic exploded view of the fork orientation of FIG. 7;

FIG. 9 shows an illustrative diagrammatic view of an assistive device and singular tool mount system being used to connect to a spatula;

FIG. 10 shows an illustrative diagrammatic exploded view of the assistive device of FIG. 9;

FIG. 11 shows an illustrative diagrammatic view of the adapter and adapter extender and mount system used to connect to a spatula;

FIG. 12 shows an illustrative diagrammatic exploded view of a the adapter and adapter extender and mount system of FIG. 11;

FIG. 13 shows an illustrative diagrammatic view of a multitool base-mount attached to a wheelchair arm with the adapter extended used to latch on the various objects;

FIGS. 14A-14B show illustrative diagrammatic exploded views of a the multitool base-mount attached to a wheelchair arm of FIG. 13;

FIGS. 15A-15C show illustrative diagrammatic views of a multitool adapter system connected to a wheelchair arm;

FIGS. 16A-16D show illustrative diagrammatic exploded views of the multitool adapter system of FIGS. 15A-15C connected to a wheelchair arm;

FIG. 17 shows an illustrative diagrammatic view of a multitool base-mount system connected to a bedside rail;

FIGS. 18A-18C show illustrative diagrammatic views of a singular base-mount system in the illustrative example showing the use of a toothbrush attachment;

FIGS. 19A and 19B show illustrative diagrammatic exploded views of the system of FIGS. 18A-18C;

FIGS. 20A-20E show illustrative diagrammatic views of a multitool base-mount system attached to a table with utensils and a cup nested on the mounts;

FIGS. 21A and 21B show illustrative diagrammatic exploded views of the multitool base-mount system according to FIGS. 20A-20E;

FIG. 22 shows an illustrative diagrammatic view of a wide-grip tool nested on a singular base-mount with a user's hand around the tool;

FIG. 23 shows an illustrative diagrammatic view of a wide-grip tool removed from the base-mount by the user;

FIG. 24 shows an illustrative diagrammatic view of a side attachment of the universal tool mount;

FIG. 25 shows an illustrative diagrammatic view of an adapter latched onto the universal tool which is nested on the base-mount;

FIG. 26 shows an illustrative diagrammatic view of a side view of the latching mechanism of the adapter, universal tool mount, and base-mount;

FIG. 27 shows an illustrative diagrammatic view of a latching mechanism of the adapter, universal tool mount, and base-mount;

FIG. 28 shows an illustrative diagrammatic view of the universal tool mount removed from the base-mount by the user;

FIGS. 29A-29F show illustrative diagrammatic views the adapter;

FIGS. 30A-30D show illustrative diagrammatic views of an angled connector;

FIGS. 31A-31D show illustrative diagrammatic views of a double-sided universal connector;

FIGS. 32A-32D show illustrative diagrammatic views of the cup holder connector;

FIGS. 33A-33E show illustrative diagrammatic views of the weighted base- mount;

FIGS. 34A-34C show illustrative diagrammatic views of the rotary base-mount;

FIGS. 35A-35D show illustrative diagrammatic views of an alternative illustrative aspect using a spring latch mechanism;

FIGS. 36A-36B show illustrative diagrammatic views of an alternative aspect using a base-mount and rod;

FIG. 37 shows an illustrative diagrammatic view of an adaptor and universal connector that includes one or more magnets; and

FIG. 38 shows an illustrative diagrammatic view of the adapter with a tapered entry for the vertical projections;

The drawings are shown for illustrative purposes only.

### DETAILED DESCRIPTION

A problem to be solved by the present invention is to provide a user with limited hand function the ability to independently attach, use, and detach securely to everyday items and to store items for later use.

The assistive adapter device and mount system is a collection of objects as shown in the accompanying drawings that allow a user with limited hand mobility to independently attach, use, and detach to various everyday objects. Everyday objects such as utensils, pens, kitchenware, etc. are commonly placed on tables, beds, in pockets, and elsewhere which make them difficult to reach and grasp for an individual with limited hand mobility.

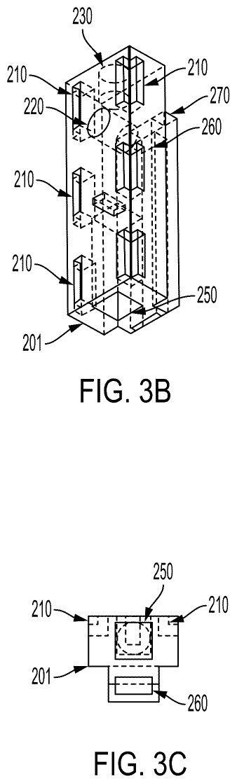

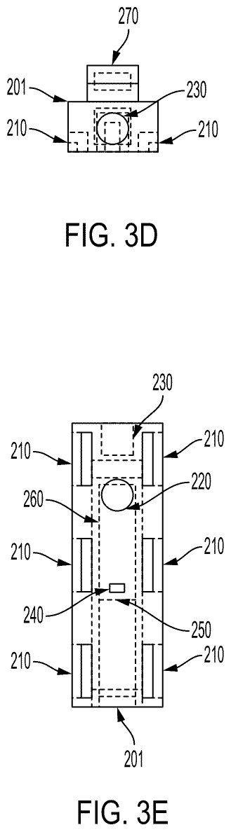

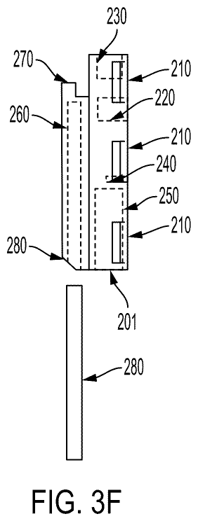

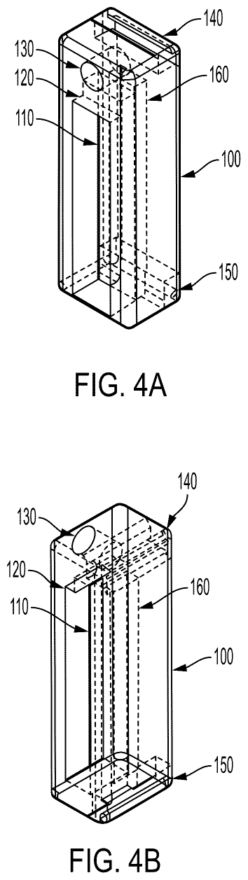

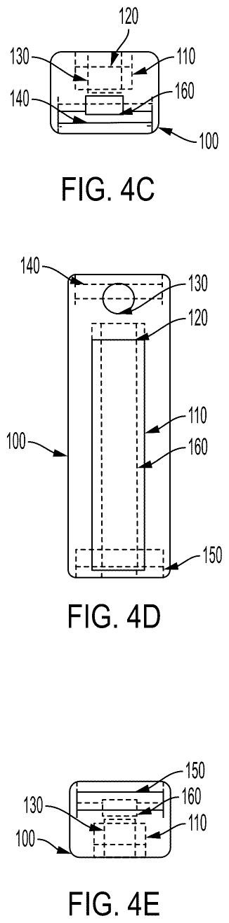

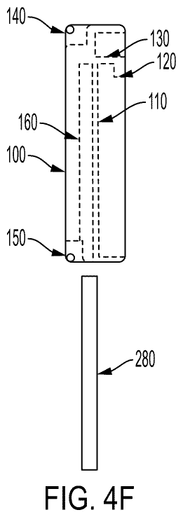

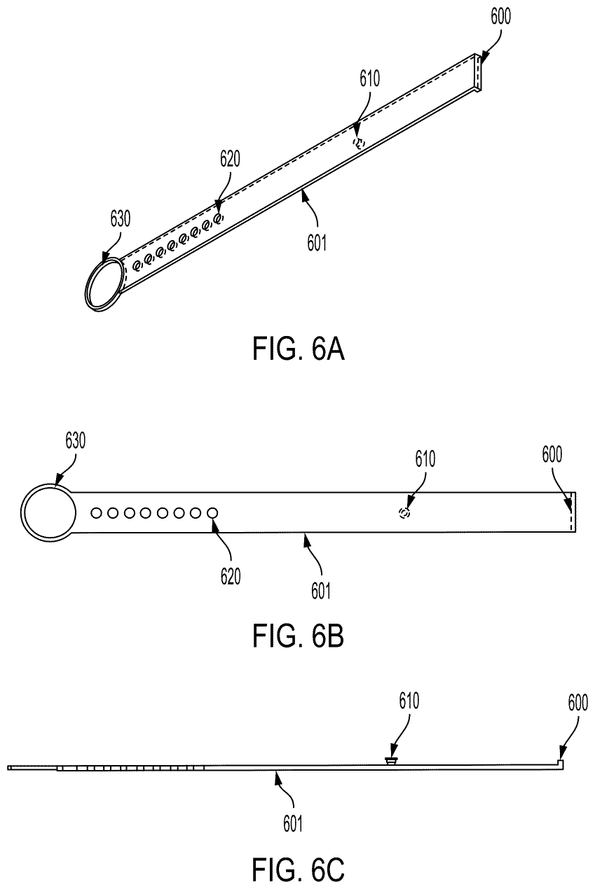

With reference to FIGS. 4A-4F, to provide the user with the ability to attach to and use various everyday objects, an adapter 100 is shown. FIGS. 4A and 4B show the adapter 100 in a perspective view, FIG. 4C shows the top view, FIG. 4D shows the front view, FIG. 4E shows the bottom view, and FIG. 4F shows a side exploded view. The adapter 100 has a female cavity 110, and an adapter cavity 140 on the top side, as will be described in more detail herein below. The adapter 100 is secured to the palm side of a user's hand (or other extremity such as an amputee's extremity) via the adapter strap, 601, seen in FIGS. 6A-6C. The adapter 100 is what allows the user to interchange tools due to its magnetic and mechanical latching features. The adapter 100 is able to latch onto various tools such as a universal connector 201 (also referred to herein as a universal tool mount), seen in FIG. 3A, via magnetic and mechanical latching features. Either the adaptor or the universal connector may include a latch with the latch cavity being provided by the other of the adaptor and the universal connector.

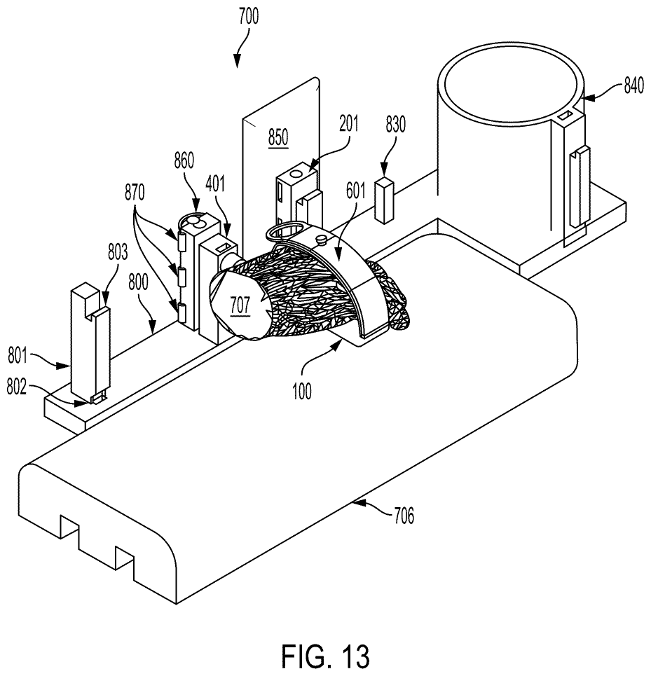

The adapter strap, 601, seen in a perspective view in FIG. 6A, is permanently attached to the adapter via the adapter strap end extruded feature, 600. This extruded feature is fit into the adapter cavity 40. As shown in the front view of FIG. 6B and the side view of FIG. 6C, and with reference to FIGS. 4A-4E, one side of the adapter strap is wrapped around adapter feature 150, which allows the adapter strap to be adjustable. The adapter strap can be secured to the user's hand (or other extremity) by holding the ring feature, 630, of the adapter strap and pulling it upwards to tighten. Once it is at the appropriate tightness around the user's hand (or other extremity), the male locking extrusion, 610, on the adapter strap can be pushed into one of the adapter strap's holes, 620. Tightening the strap and securing it via 610 mating with 620, can be done through simply putting the user's hand through the strap and pulling on the ring, 630. By having a ring, 630, on the end of the strap, users with limited hand mobility can use a finger of the non-adapter hand to pull, tighten, and secure the adapter strap. Examples of the adapter 100 secured to the user's hand via the adapter strap can be seen in FIGS. 13-21B.

The adapter 100 comprises a female cavity 110, seen in FIGS. 4A-4F, on the palmar face of the adapter, facing away from the user's palm, which couples with the latch projection 270 of the universal tool mount 201, seen in FIG. 3A. The latch projection 270 of the universal tool mount 201, provides a mechanical force against the walls of the cavity of the adapter 100 allowing for a secure connection. Referring now to both FIG. 4A and FIG. 3A, a top surface 120 within the cavity 110 on the adapter 100 engages with the latch projection 270 of the universal tool mount 201 when inserted, thereby latching the universal tool mount and adapter mechanically. This allows the user to carry heavier objects since the top surface 120 is engaged to the latch projection 270 of the universal tool mount 201. Furthermore, a magnet is placed in the cavity 160 within the adapter 100, which is attracted to a magnet placed within the cavity 260 within the universal tool mount 201. This magnetic attraction provides an additional secure latching connection between the adapter 100 and universal tool mount 201.

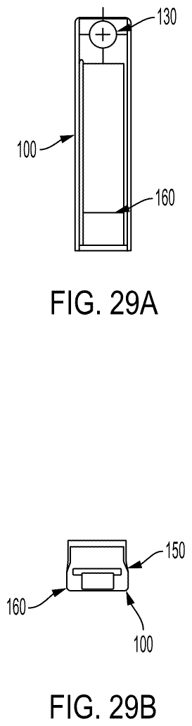

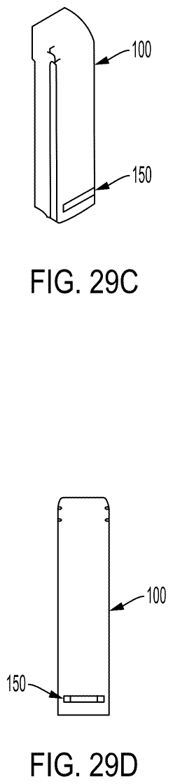

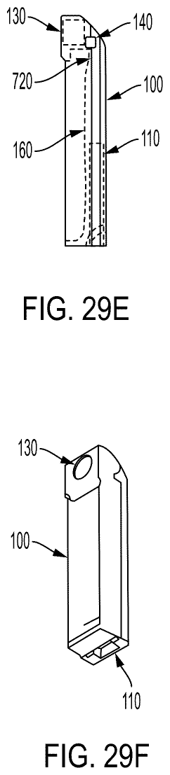

The adapter 100 can also be seen in FIGS. 29A-29F in an alternative illustrative aspect. FIG. 29A shows the adapter 100 with the cavity 130 and the cavity 160 to receive the universal tool mount 201 as described herein. A bottom view of the adapter 100 is shown at FIG. 29B with the cavity 160 and the wrist strap feature 150. A perspective view of the adapter 100 is shown at FIG. 29C and the back view of the adapter 100 is shown at FIG. 29D. FIG. 29E shows a cutaway side view of the adapter 100 with the cavity 160 shown here with a revised angled recess 720 so the adapter can easily be pivoted out of the latched position when connected to another part in the system such as the universal connector. A lower perspective view of the adapter is shown at FIG. 29F.

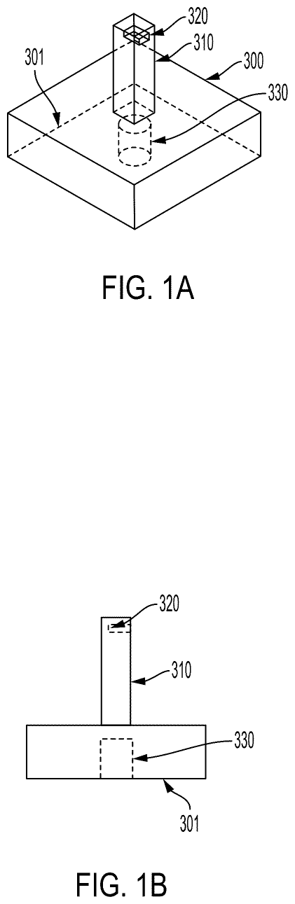

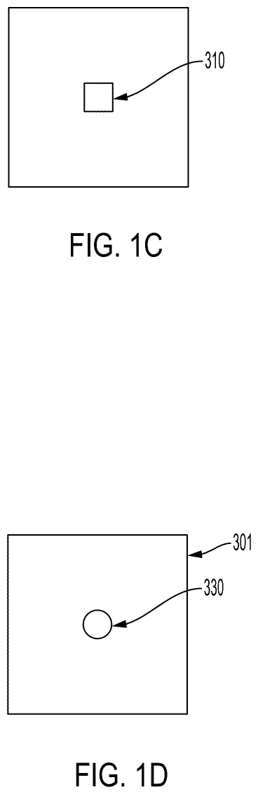

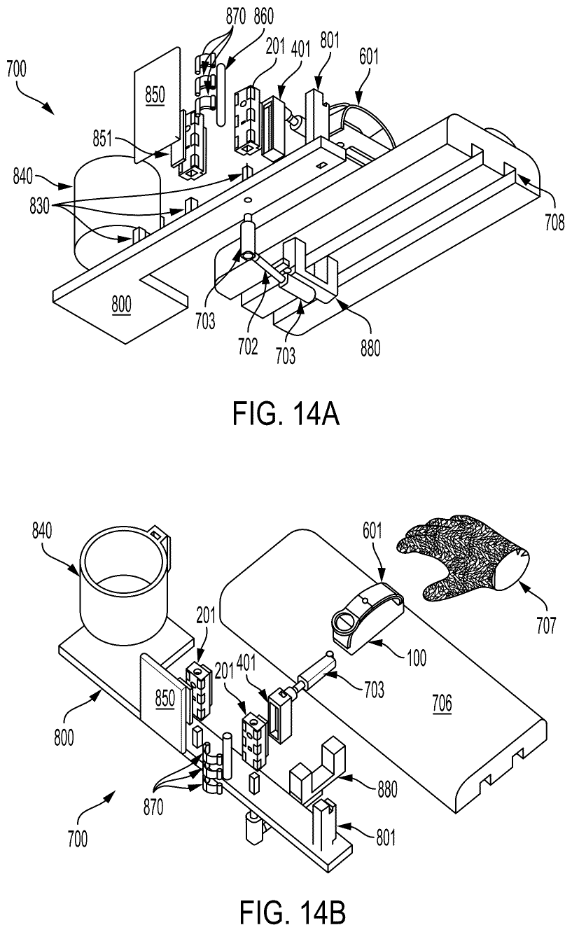

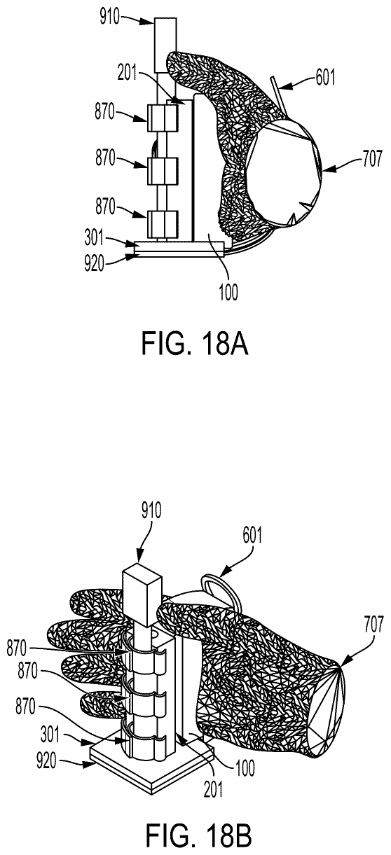

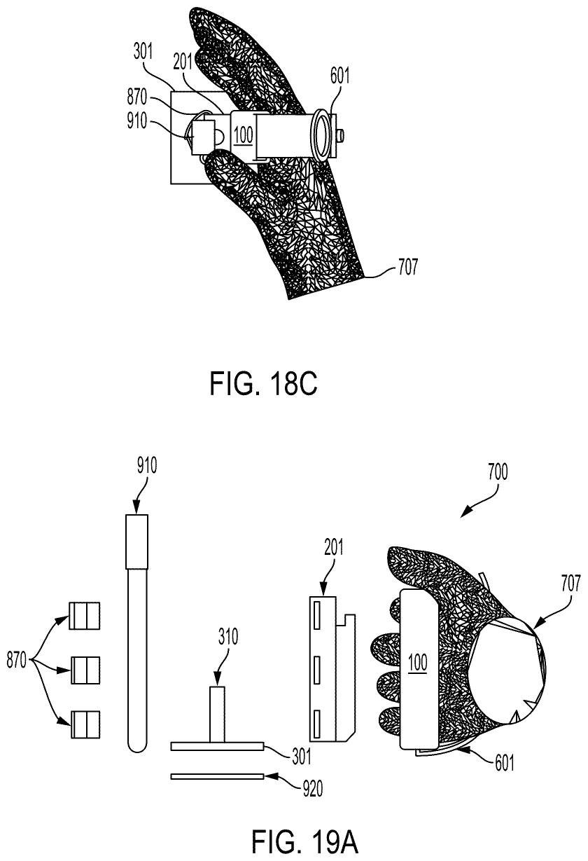

The universal tool mount 201 is described with reference to FIGS. 3A-3F, and allows users to attach various everyday objects to tool such as utensils, kitchenware, bathing devices, grooming devices and more. FIG. 3A shows a perspective view of the universal tool mount 201 where tools can be attached with loop-lock or Velcro-like straps using slotted holes 210, or such tools inserted into top hole 230, side hole 220, or other suitable attachment method. The latch projection 270 engages with the adapter 100, as herein described. With further reference to FIG. 3A, rear perspective view at FIG. 3B, bottom view at FIG. 3C, top view at FIG. 3D, rear view at FIG. 3E, and side view at FIG. 3F, features are shown to describe the operation of the adapter 100 to engage and secure the universal tool mount 201. Vertical projections 280 insert into a recessed cavity 260. Magnet 240 is strategically placed to attract to the corresponding magnet on the adapter 100. During operation, the tool is oriented in the vertical position for case of latching with the adapter from the user. To maintain this vertical position of the universal tool mount when not being in use, the singular base-mount 300, shown in FIGS. 1A-1D. FIG. 1A is a perspective view of the singular base mount 300, FIG. 1B is a side view, FIG. 1C is a top view and Figure ID is a bottom view of the singular base mount 301. The singular base mount has a vertically extending portion 310 extending upwards so that the universal tool mount 201 can engage and insert into the universal tool mount cavity 250. In accordance with certain aspects, the vertically extending portions may be detachable but when present are secured to the base mount. The singular base-mount 301 can be secured to a table or work surface using adhesive, for example, using double sided adhesive 920 as shown in FIG. 18A-18C. Also, the thread opening of the singular base-mount 330 allows the attachment of a tripod type threaded ball joint which can be further attached to a socket and clamp, similar to the attachment of alternate embodiments used with wheelchair arms or bedside rails as shown with reference to FIGS. 14A and 14B.

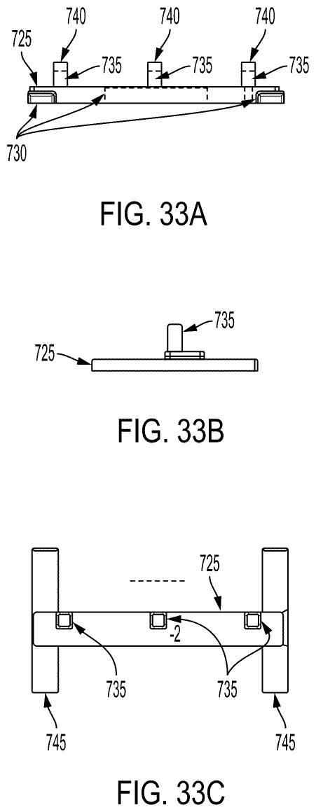

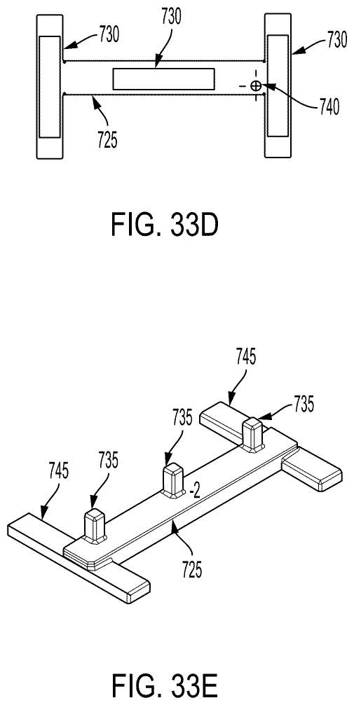

A variation of the singular base mount is shown at FIGS. 33A-33E. FIG. 33A shows a longitudinal side section view of a weighted base mount 725. FIG. 33B shows a side view of the weighted base mount 725. FIG. 33C shows a top view of the weighted base mount 725. FIG. 33D shows the bottom view and FIG. 33E shows a perspective view of the weighted base mount 725. The weighted base-mount 725 includes base mount extrusions 735 and has stabilizing arms 745 and weight cavities 730 for weights so that it could be placed on a surface without an adhesive and stay in place while the user inserts objects on and off of the extrusions 735. It is still capable of being connected to a universal joint with a threaded insert cavity 740 on the bottom or adhered to a surface via double sided adhesive or hook and loop straps.

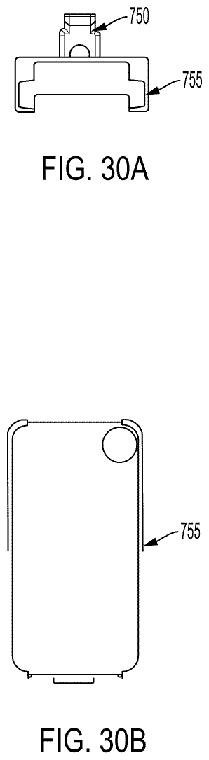

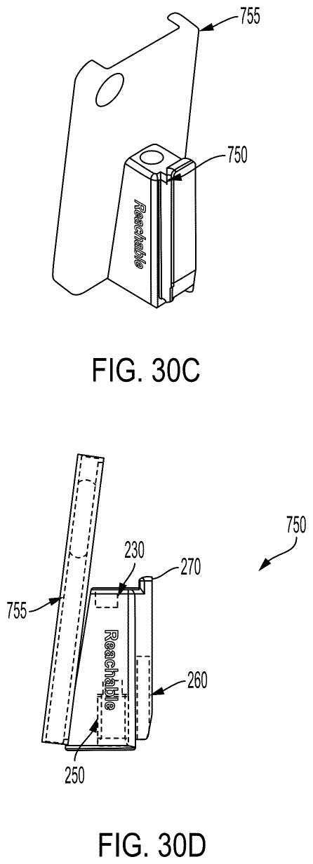

An angled tool mount 750 is shown at FIGS. 30A-30D. FIG. 30A shows a top view of the angled tool mount 750 with an attached phone case 755, FIG. 30B shows a front view of the angled tool mount showing the phone case 755, FIG. 30C shows a rear perspective view of the angled tool mount 750. FIG. 30D shows a side view of the angled tool mount 750 with the phone case 755 attached at an ergonomically optimized angle with the top hole 230, magnet cavity 260, and receptacle cavity 250 shown in outline. The angled tool mount 750 has similar functionality as the universal tool mount 201 whereas it can be placed on a base-mount 301 when not used by a user, has a magnet cavity 260 and latch projection 270 to engage in the adapter cavity. An object such as a phone case 755 can be attached to the angled tool mount 750 via adhesive. The face of the angled tool mount which connects to the phone is angled to provide the user with optimal ergonomic viewing of the object when engaged with the adapter 100 coupled to the user's hand (or other extremity).

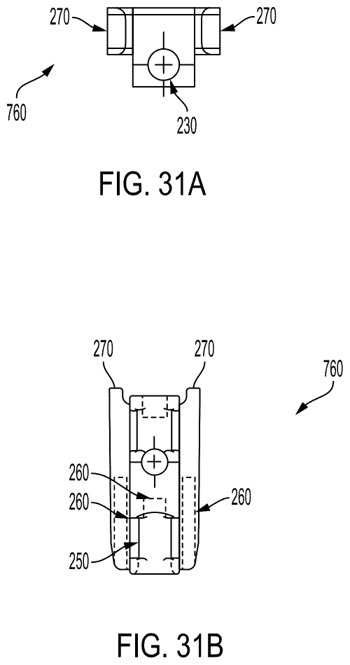

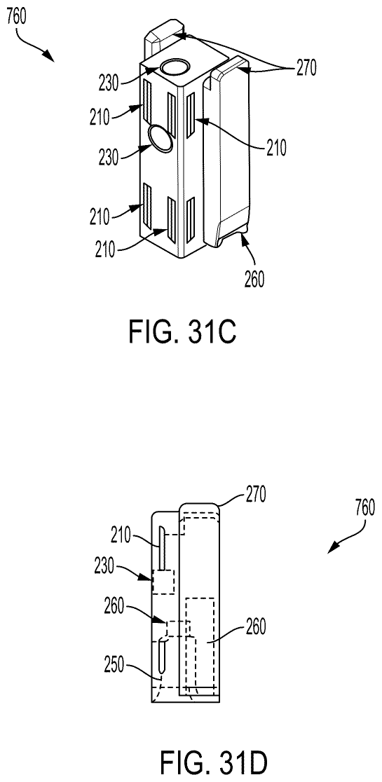

A double-sided universal tool mount 760 can be seen in FIG. 31A-31D. FIG. 31A shows a top view of the double-sided universal tool mount 760 showing a threaded hole 230 for which to attach a tool and latch projections 270. FIG. 31B shows a front view of the double-sided universal tool mount 760 showing each side having a latch projection 270 and magnetic cavities 260. A single receptacle 250 for the vertical projections of the base-mount is provided. FIG. 31C shows a rear perspective view of the double-sided universal tool mount 760 with both latch projections 270 with strap slots 210 and two threaded holes 230 for attachment of tools. The magnetic cavity 250 provides a latch function with the adapter as described herein. FIG. 31D shows a side view of the double-sided universal tool mount 760 with a latch projection 760 and strap slots 210 shown with threaded holes 230, magnetic cavity 260, and receptacle 250 in outline. The double-sided universal tool mount 760 has similar function as the universal tool mount 201, but has the latching mechanism on each side so that someone could attach to the double sided tool mount and use an object one way and then place the tool mount on a base-mount, turn the tool mount and attach to the other latching mechanism to use the object in another orientation. This could be helpful, for example when one is brushing their teeth and needs to change the orientation of the toothbrush.

A simple illustration of what an example of the adapter device and mount system can be seen in FIGS. 18A-18C. FIG. 18A is a side view and FIG. 18B is a perspective view showing the user's hand (or other extremity) 707 secured to the adapter 100 via the adapter strap, 601. With the adapter 100 secured to the palmar side of the hand latched to the universal tool mount 201, which is nested onto the base-mount 301 which is adhered to a table via double sided adhesive 920 (or suitable means for attachment such as a suction cup or loop-lock material such as Velcro). The universal tool mount 201 in the illustrative embodiment according to FIGS. 18A-18C is shown with a toothbrush 910 attached using three Velcro straps 870. The universal tool mount 201 can connect to various other objects in various methods.

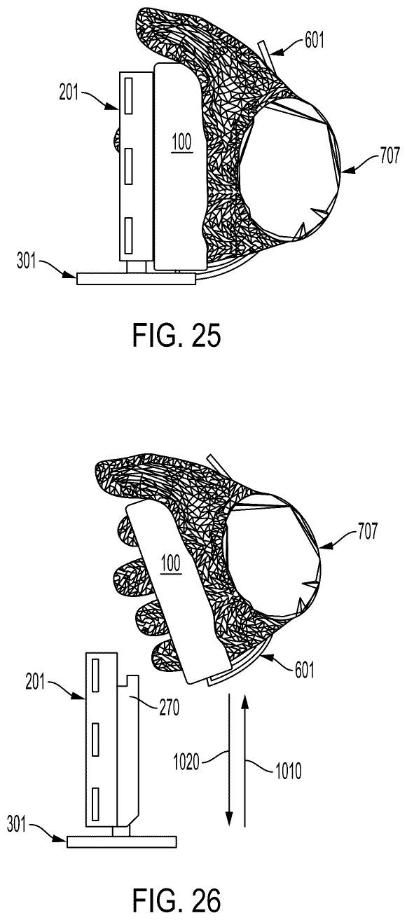

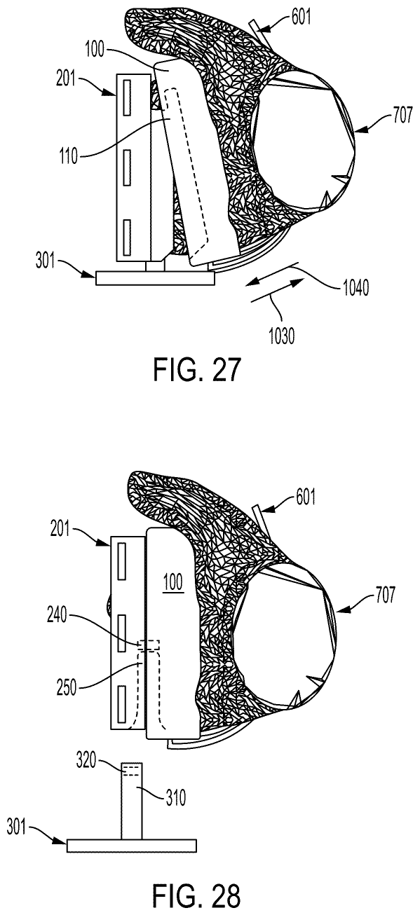

The method of latching the adapter to the universal tool mount is illustrated in FIGS. 25-28. FIG. 26 illustrates the adapter 100 secured to the palm side of the hand (or other extremity) 707 via the adapter strap 601. In this position, the user can move freely with the adapter 100 and independently choose which tool to latch to. Once the user chooses a tool and the corresponding universal tool mount 201, the user can move their arm in a downward slightly outward (pronation) motion, 1020, to latch onto the universal tool mount 201 which is nested onto the base-mount 301 as shown in FIG. 26. FIG. 27 illustrates the user mechanically latching the cavity 110 of the adapter to the latch projection 270 (FIG. 26) of the universal tool mount 201. Once the latch projection 270 the universal tool mount 201 is inside of the cavity 110 of the adapter 100, the user can make a slight supination movement, 1040, to fully latch the two parts together, engaging both the magnetic and mechanical latching function. FIG. 25 illustrates the adapter 100 and universal tool mount 201 latched together while the universal tool mount 201 is still nested on the singular base-mount 301. The adapter 100 is secured to the universal tool mount 201 and the user can now lift their hand (or other extremity) upwards, as shown in FIG. 28, freeing the universal tool mount 201 from the singular base-mount 301. FIG. 28 illustrates the user independently using the universal tool mount 201 which can be connected to various everyday objects. Once the user decides they want to detach to the universal tool mount 201 they are using, they bring their hand (or other extremity) over the singular base mount 301, seen in FIG. 28, and make a downwards motion to nest the universal tool mount 201 onto the vertically extending portion 310 of the singular base-mount 301. FIG. 27 illustrates the user detaching the adapter from the universal tool mount by making a slight supination movement, 1030. This disengages most of the magnetic latching properties allowing the user to make an upward motion 1010, as shown in FIG. 26 to disengage the rest of the mechanical latching properties. The universal tool mount 201 remains on the vertically extending portion 310 partially due to a magnetic attraction between a magnet inserted into the base-mount cavity 320 and a small magnet 240 placed in the cavity 250 that receives the vertically extending portion 310 in the universal tool mount 201. These two magnets create a small attraction which helps the universal tool mount 201 remain on the base-mount 301 when the user wants to detach the adapter, but also allows the user to the easily detach the universal tool mount 201 from the base-mount 301 due to its small magnetic force compared to the mechanical and magnetic latching of the adapter 100 and universal tool mount 201. Once the hand 707 has moved up over the universal tool mount, seen in FIG. 26, the user may independently latch onto other objects.

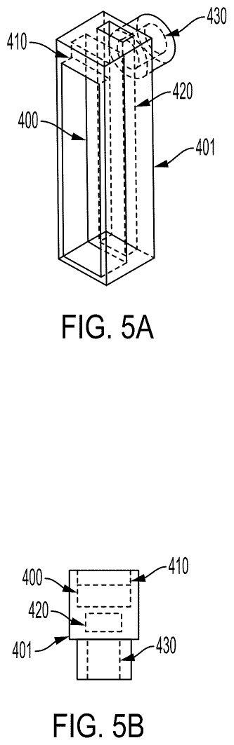

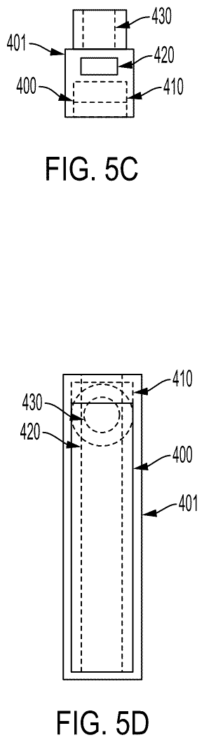

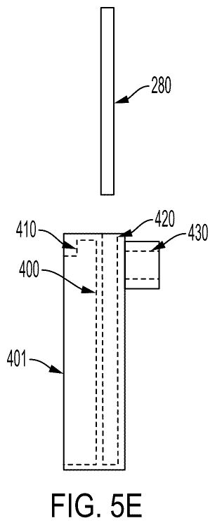

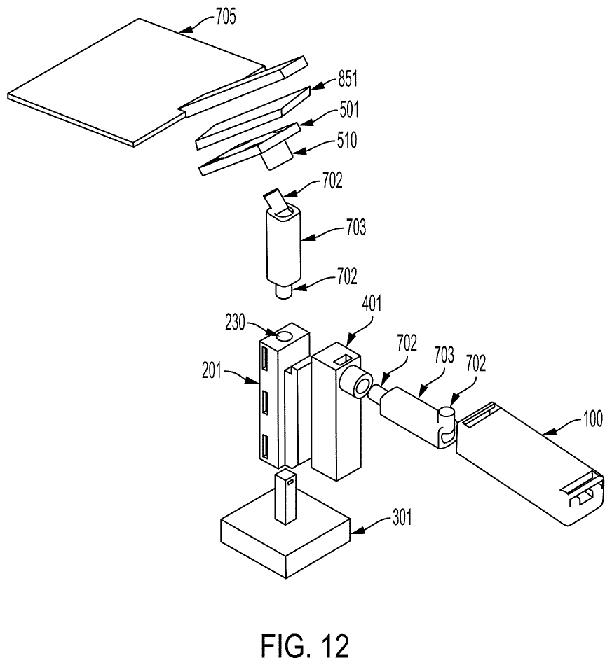

If a user with limited mobility also has their hand (or other extremity) fixed in a pronated position an adapter extender 401 as shown in FIGS. 5A-5E can be attached to the adapter 100 via a universal joint such as ball joints 702 and a socket 703 as shown in FIG. 12. The adapter 100 has a hole 130 (as shown in FIGS. 4A-4F) into which a ball joint 702 can be threaded. The ball joint 702 is connected to a socket 703 which connects another ball joint 702 that can thread into the threaded opening 430 of the adapter extender 401. This provides customizable angles to allow the user with a hand in a fixed position to use the adapter extender in the same way the adapter is used. The adapter extender 401 according to FIGS. 5A-5E has a mechanical cavity, 410, and magnet cavity, 400, which are functionally equivalent to the latch cavity 110 and magnet cavity 160 of the adapter 100.

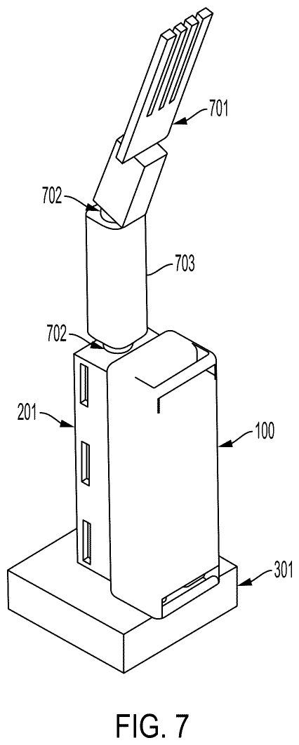

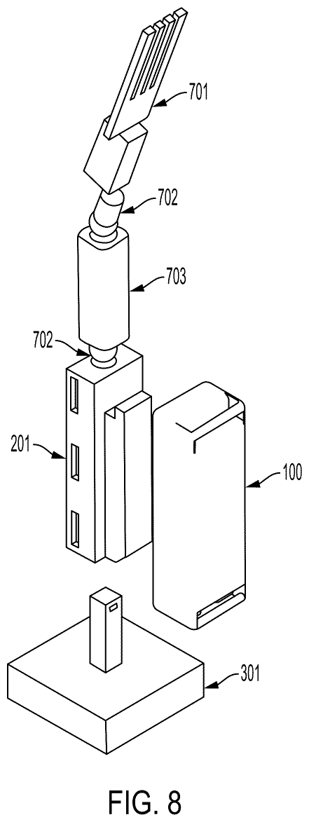

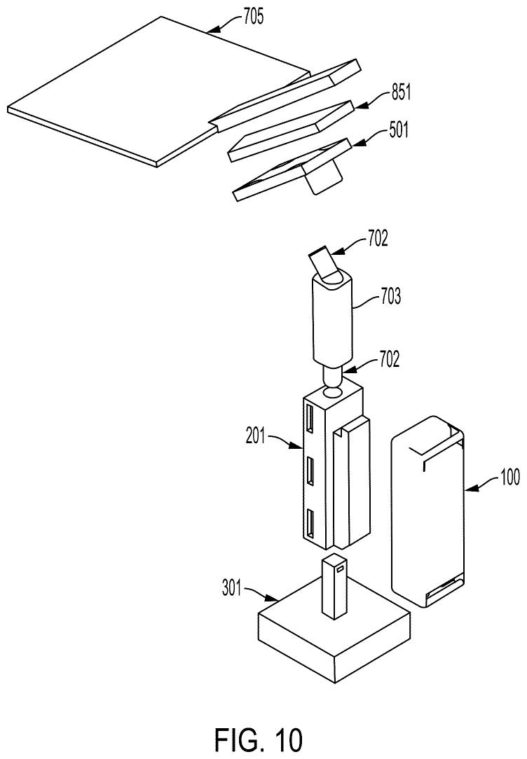

The universal tool mount 201 has several features that permit external attachment to everyday objects. The features include the hole 230 at the top of the universal tool mount 201 seen in FIG. 3A, allowing a connector such as a threaded ball joint 702 to be inserted. The ball joint 702 can be connected to another ball joint via a socket 703 so that a utensil such as a fork 701 (shown in FIGS. 7 and 8), or a spoon 940 can be connected as well as other parts. The ball and socket connection from the utensil to the universal tool mount 201 allows for customization of the utensil angle relative to the universal tool mount 201. Another feature is the side threaded hole 220 on the front side of the universal tool mount 201 into which a ball joint 702 can be threaded. This may allow for a similar attachment of a utensil if the user wanted the utensil to face perpendicular from the user's palm. Yet another feature for external attachment includes the strap holes 210 that permits a tool to be strapped to and frictionally held against the front face of the universal tool mount 201. Additionally, the front face of the universal tool mount 201 can utilize a double sided adhesive 851 (similar to double sided adhesive 851 shown in FIG. 10) can be placed in between the front face of the universal tool mount and an object.

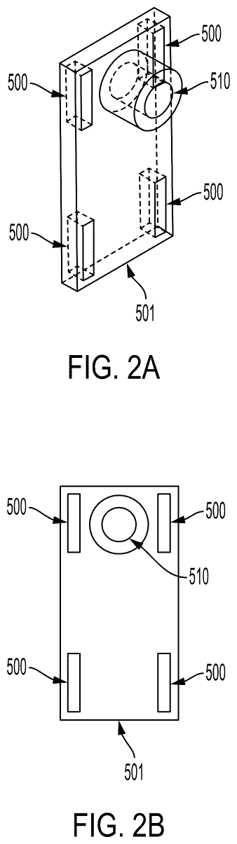

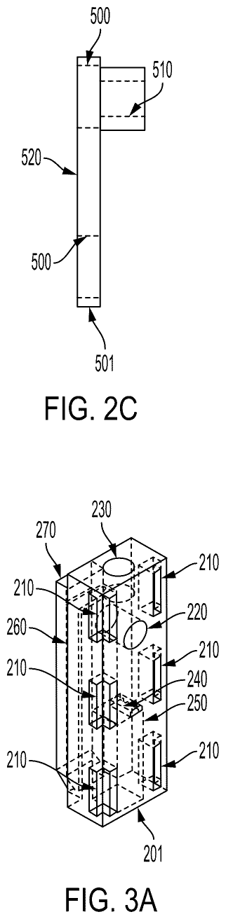

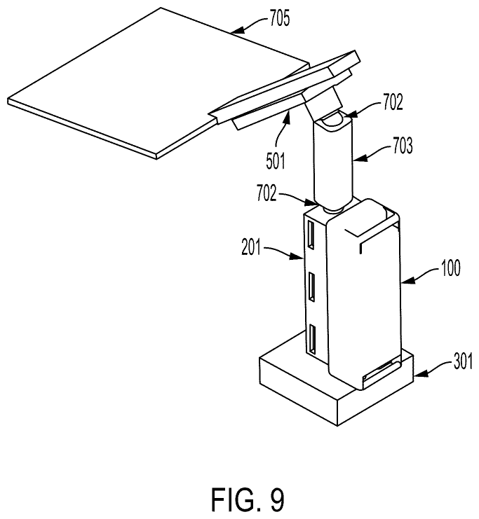

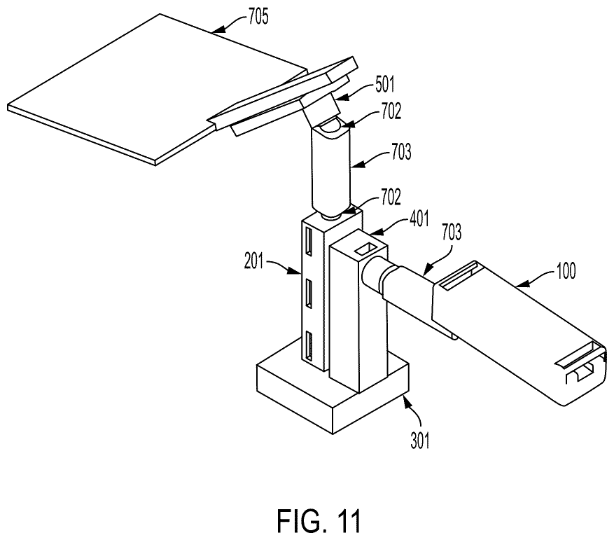

The top threaded hole 230 of the universal tool mount 201, as shown in FIG. 3A, can receive a threaded ball joint 702 for attaching tools, it can also be used for attaching the tool extender 501 as shown in FIG. 2A-2C. The tool extender 501 is shown in perspective view at FIG. 2A, front view in FIG. 2B, and side view in FIG. 2C, which includes the tool extender strap slots 500, tool extender threaded hole 510, and tool extender back side 520. The tool extender 501 is an extension of the universal tool mount so that an object which needs to be in a non-upright position can be correctly oriented. For example, if a spatula was attached to the universal tool mount 201, it would be in a vertical orientation and therefore difficult to use, particularly if the user has difficulty with hand (or other extremity) and arm mobility. With reference to FIGS. 11 and 12, a correctly oriented assembly can be made by attaching the tool extender 501 to the universal tool mount 201 via ball joints 702 and ball sockets 703 through threaded hole 230 of the universal tool mount 201, into the threaded hole 510 in the tool extender 501. This allows for a customizable angle so that objects can be correctly orientated as seen in FIG. 9. The tool extender 501 has two of the universal tool mount's external attachment features such as Velcro strap holes 500 and the back face to allow for double sided adhesive 851 as shown in FIG. 10.

FIG. 13 depicts an accessibility system 700 using the adapter 100 and multiple tool base-mount 800 shown with a wheelchair armrest 706. FIG. 13 is an example of what the multitool base-mount 800 may look like when connected to a wheelchair armrest 706 and a possible tool configuration. As shown in FIG. 13, the tools mounted on the multiple tool base-mount 800 include a cup 840, a phone 850, and a stylus 860. The cup 840 has the elements of the universal tool mount 201 integrated into it, with the magnetic and mechanical locking mechanism as a part of the cup 840. The user can directly use the adapter 100 or adapter extender 401 to latch onto the cup 840 and independently use the cup 840. The phone 850 is connected to a universal tool mount 201, which is positioned on a vertical projection 830. There is also a stylus 860, which is connected to a universal tool mount 201 via three Velcro straps 870. The user's hand (or other extremity) 707, in this example, is using the adapter extender 401 which is able to latch onto any of the tools for the user to independently use. Also shown is an adapter donning and doffing station 801, where the user can attach or detach the adapter 100. The adapter donning and doffing station 801 is similar to the universal tool mount 201 because it has the same mechanical and magnetic latching system, but it is attached to the multitool base-mount 800. A hole 802 is provided for a magnet with opposite polarity of the magnet used in the adapter 100 or adapter extender 401. A mechanical latching feature 803 is provided for a secure coupling of the adapter 100 or adapter extender 401 at adapter donning and doffing station 801. Due to the adapter donning and doffing station 801 being part of the multitool base-mount 800, it provides a more rigid coupling which allows the user to unlatch the adapter strap 601, and allow the user's hand 707 to slip out of the adapter device and strap. The user is then able to use their hand 707, for other daily activities while having the adapter device correctly orientated by being secured to the adapter donning and doffing station for future use. When the user wants to use the adapter 100 again, they put their hand 707 through the loosened adapter strap 601 and can use the straps circular ring 630, as shown in FIGS. 6A-6C to tighten the strap around the user's hand for proper use.

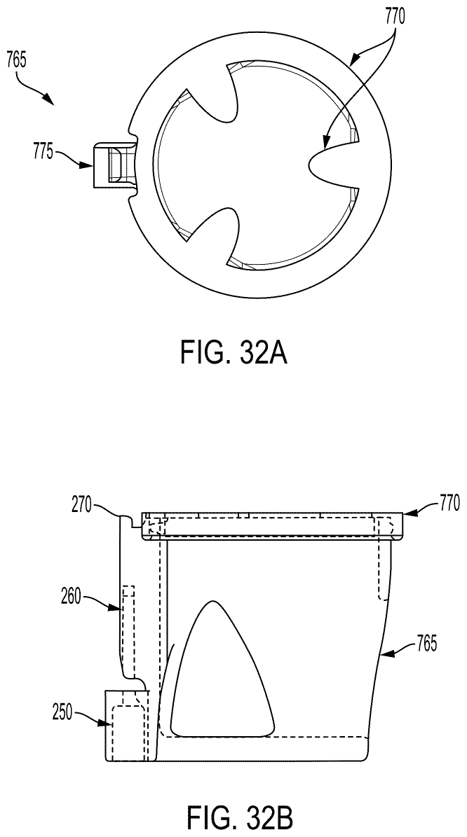

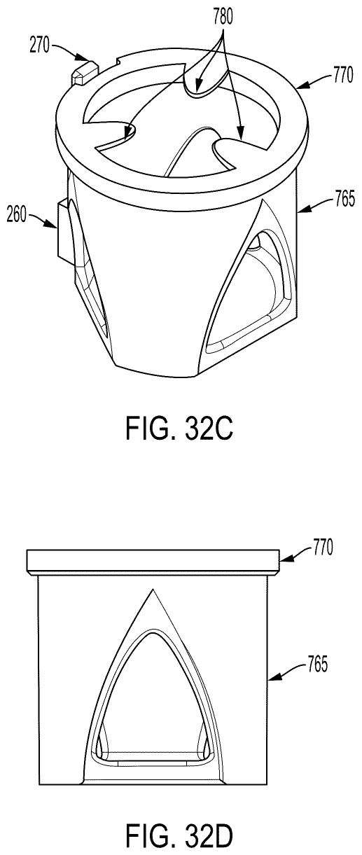

The cup 840 in FIG. 13 can alternatively be provided as a cup holder 765 as shown in FIGS. 32A-32D. FIG. 32A is a top view of the cup holder 765, FIG. 32B is a front view of the cup holder 765, FIG. 32C is a perspective view of the cup holder 765, and FIG. 32D is a side view of the cup holder 765. The cup holder comprises a removable retainer 770 that is fitted on the top of the cup holder 765 with retainer flaps 780 to keep a bottle or disposable cup in place. The body of the cup holder is used as a structure to hold a cup in place as well as have the vertical projection reception cavity 250 and latching mechanism including the latch projection 270 and magnetic cavity 260 for the adapter 100 to be latched onto.

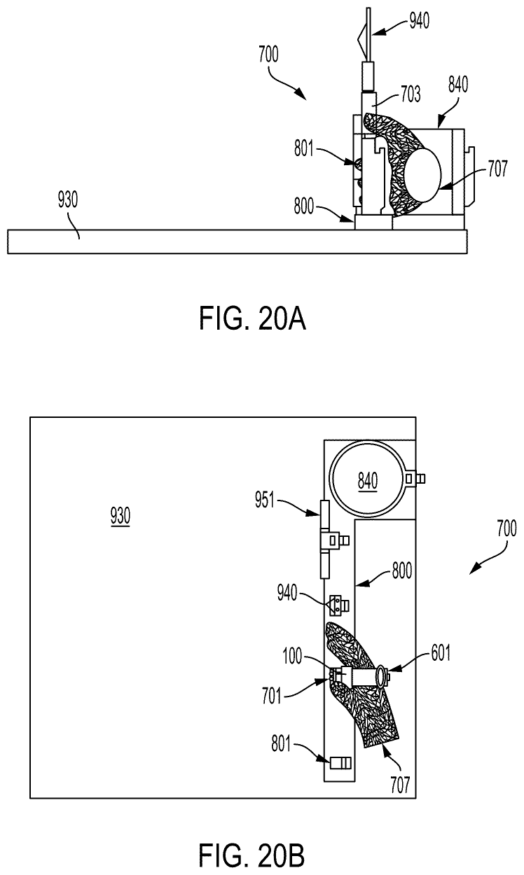

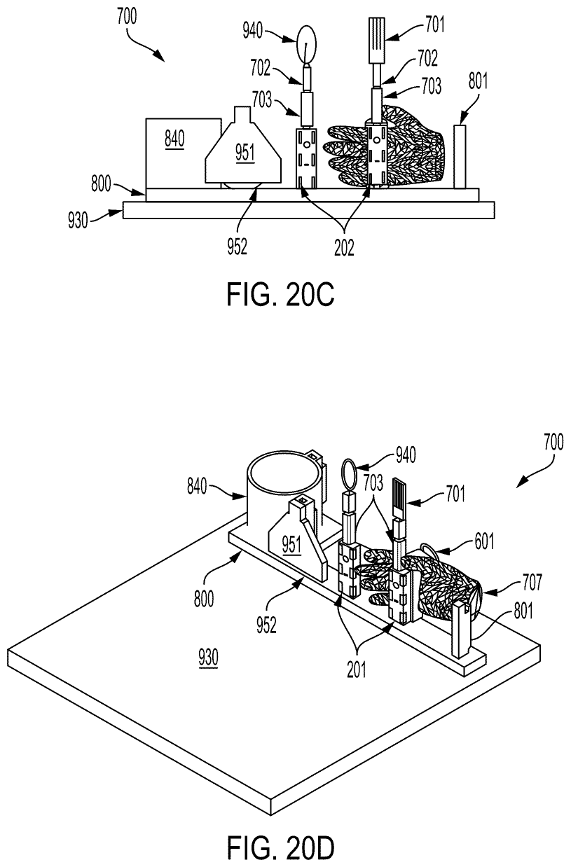

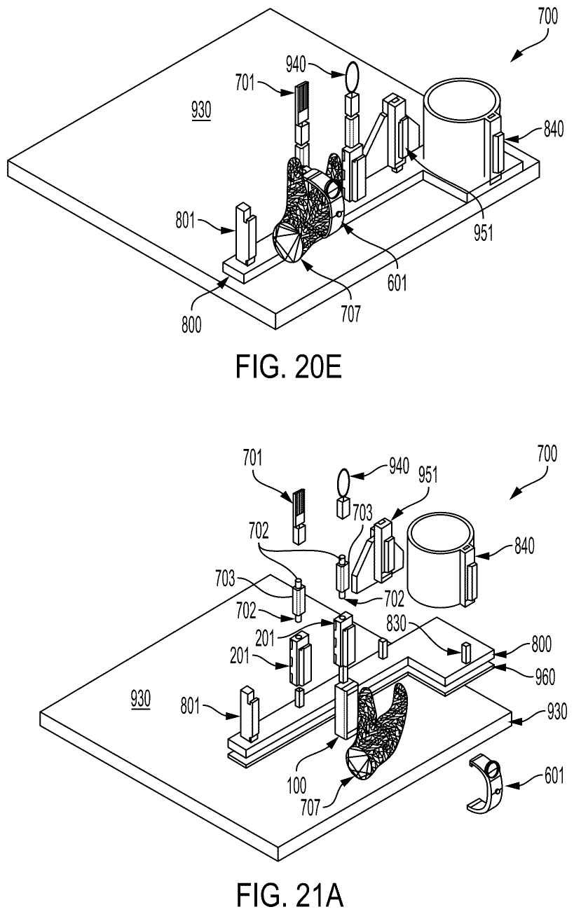

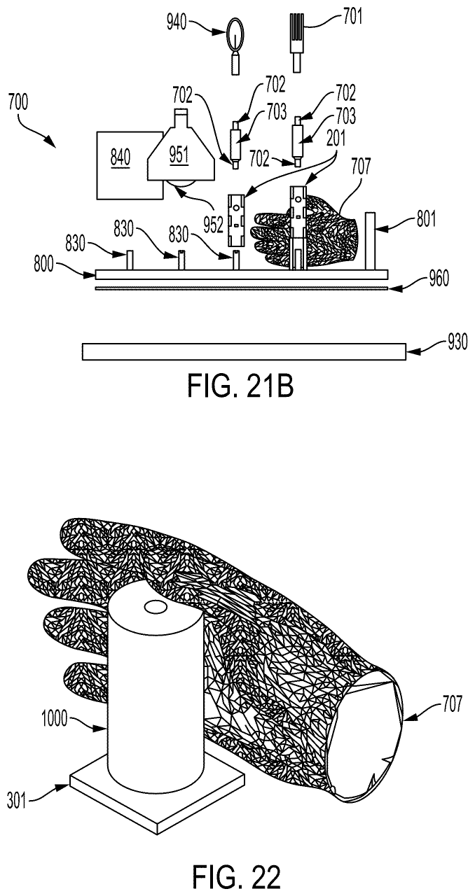

FIG. 14A is a bottom perspective view and FIG. 14B is a top exploded view of the accessibility system 700 mounted to a wheelchair arm 706. A clamp 880 is clamped to the wheelchair arm. Two ball joints 702 and corresponding sockets 703 are connected in series with one ball joint 702 connected to the clamp 880 and the other ball joint 702 connected to a threaded hole on the bottom on the multitool base mount. Since the bottom of the multiple tool base-mount 800 has an opening for a threaded ball joint 702, the multiple tool base-mount 800 can not only be mounted to various objects including a wheelchair armrest 706, but it can be adhered to any work surface. For example, as shown in FIGS. 20A-20D and FIGS. 21A and 21B, the multiple tool base-mount 800 displays a possible configuration of a stylus 860 strapped via three Velcro straps 870 to a universal tool mount 201 nested on one of several vertical protrusions 830, for tools on the multiple tool base-mount 800. FIG. 20A shows a side view of the accessibility system 700 mounted on a table-top 930. FIG. 20B shows a top view of the accessibility system 700 mounted on a table-top 930. FIG. 20C shows a back side view of the accessibility system 700 mounted on a table-top 930. FIG. 20D and FIG. 20E each show a perspective view of the accessibility system 700 mounted on a table-top 930. FIG. 21A shows an exploded perspective view of the accessibility system 700 mounted on a table-top 930 and FIG. 21B shows a back side exploded view of the accessibility system 700 mounted on a table top 930 using adhesive material 960. In each of FIGS. 20A-20E and FIGS. 21A and 21B, the strap 601 is shown in the locked position around the user's hand (or other extremity) 707 and connected to the adapter 100. The adapter 100 is connected to the adapter extender 401 via two ball joints 702 and one socket 703. This allows the user's hand 707 to be in a pronated position, but still be able to use the system to use various tools placed on the multiple tool base-mount 800. A phone 850 is connected to a universal tool mount 201 via double sided adhesive 851. Also each mounted on universal tool mounts 201 are a fork 701, a spoon 940, and a knife 951 with a knife circular blade 952. The donning and doffing station 801 is provided for the user to latch into and don, doff, or store the adapter 100 as is described herein above.

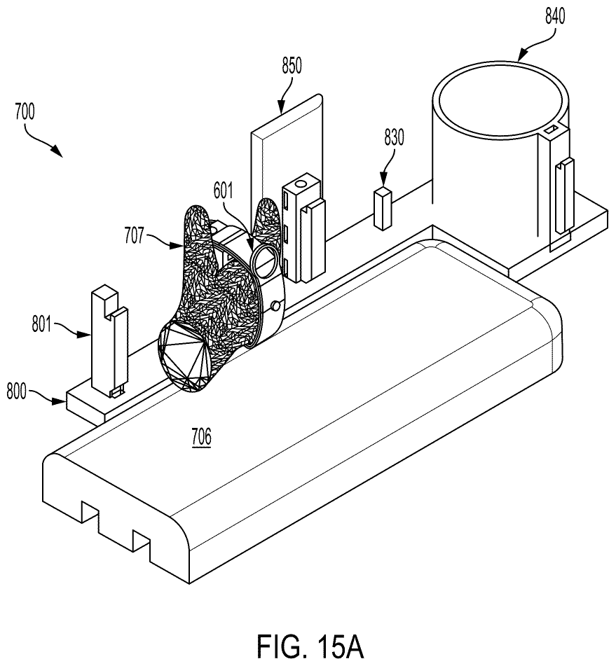

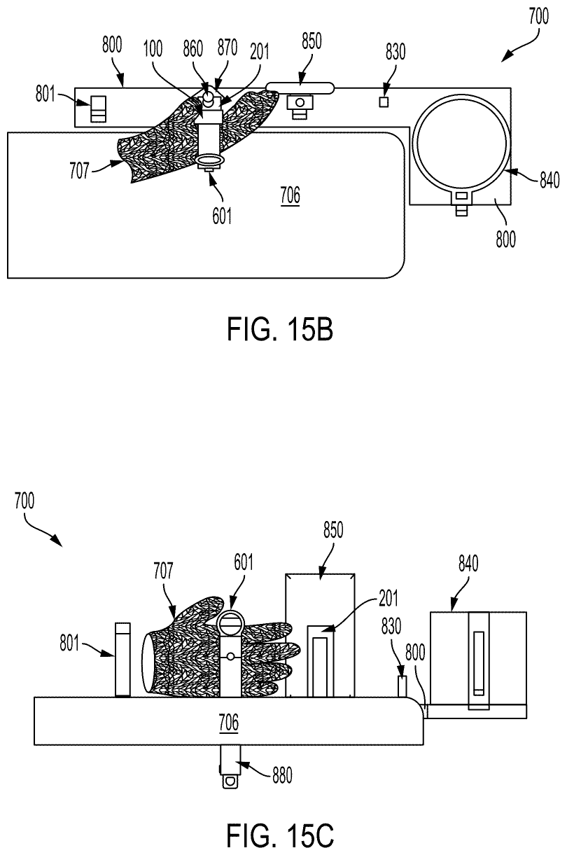

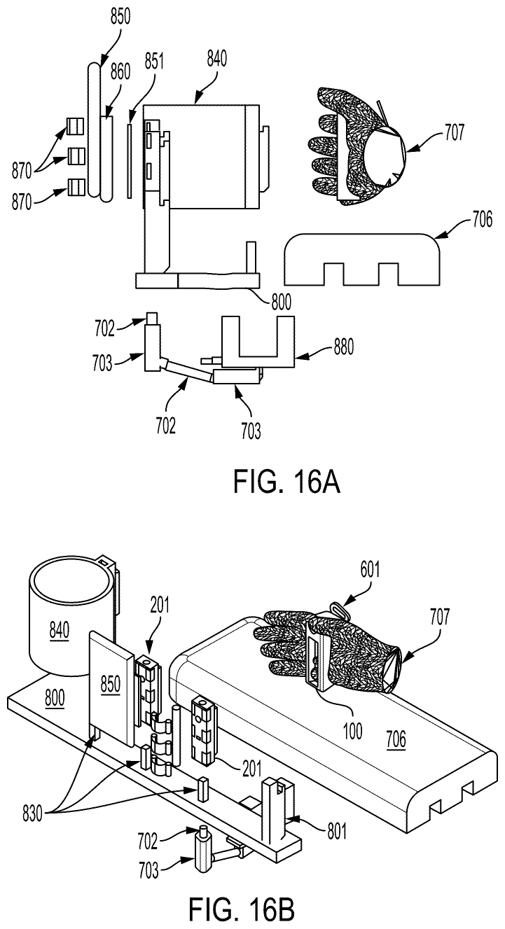

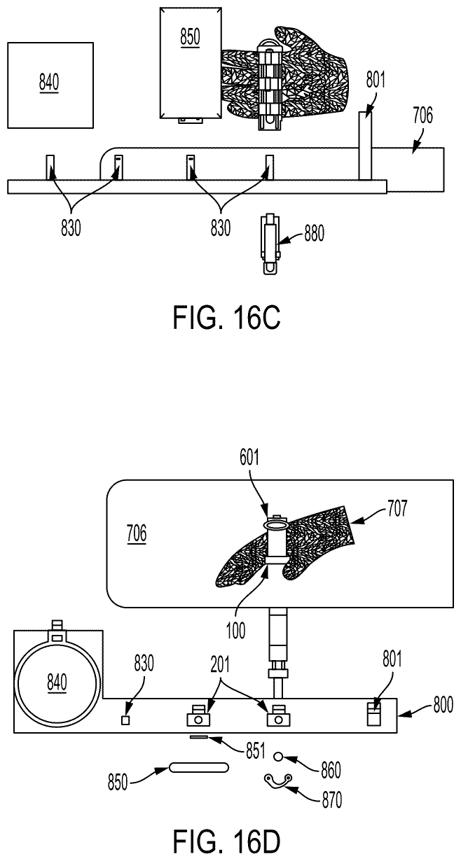

FIGS. 15A-15C shows the accessibility system 700 with the use of the adapter 100 instead of in combination with the adapter extender 401 as described above with reference to FIG. 13. FIG. 15A shows a perspective view of the accessibility system 700 attached to a wheelchair arm 706. FIG. 15B shows a top view of the accessibility system 700 attached to a wheelchair arm 706. FIG. 15C shows a side view of the accessibility system 700 attached to a wheelchair arm 706. In FIGS. 15A-15C, the user's hand 707 with the adapter strapped with strap 601 is configured to latch onto the various objects on the multiple tool base-mount 800. FIG. 16A, FIG. 16B, FIG. 16C and FIG. 16D are each an exploded view of the accessibility system 700 attached to a wheelchair arm 706 as depicted generally in FIGS. 15A-15C. The clamp 880 as well as the ball joint 702 and sockets 703, connecting the wheelchair arm 706 to the multiple tool base-mount 800 can be seen.

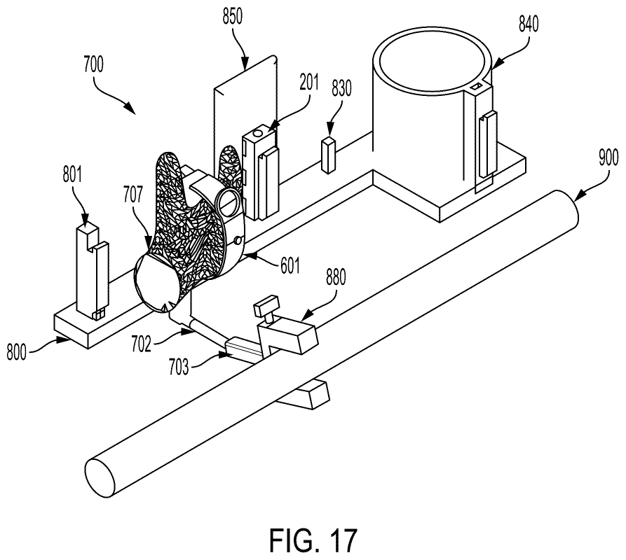

FIG. 17 shows the accessibility system 700 attached to a bedside rail 900. The multiple tool base-mount 800 and adapter 100 are shown being used when the multiple tool base-mount 800 is connected to a bedside rail 900, as is commonly required in hospital settings. The clamp 880 and ball joint 702 and socket 703, are similar to those described herein above demonstrating the flexibility of the various environments the multiple tool base-mount 800 may be clamped onto. The multiple tool base-mount 800 may be clamped onto other everyday objects other than a wheelchair arm 706 or bedside rail 900 to allow the user to interchange tools independently. The multiple tool base-mount 800 may also be adhered to a surface without the need of the clamp 880. The flexibility of the clamp 880 and cooperative ball joint 702 and socket 703 does permit the user or a caregiver to adjust the position of the multiple tool base-mount 800 so that it can be easily accessed by the user. It can also be mounted and configured on the right or left side of the user.

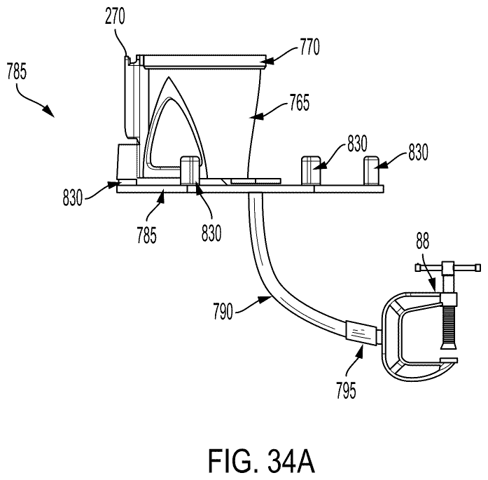

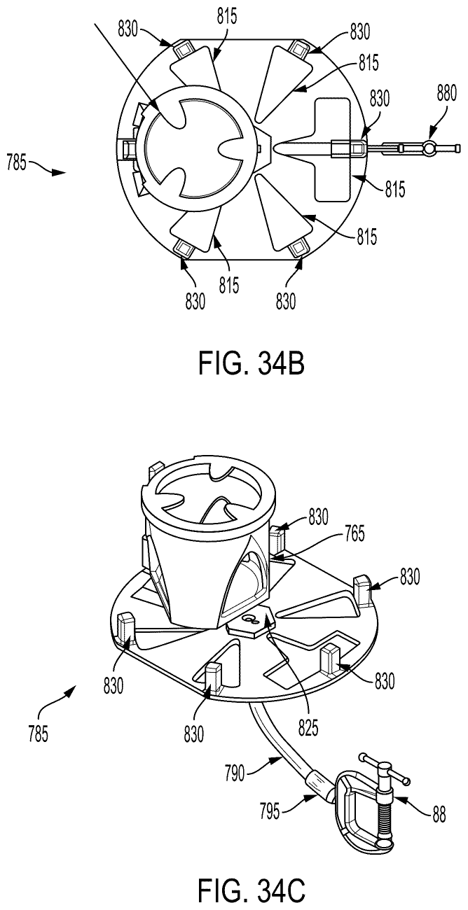

FIG. 34A shows a side view of a rotating multiple tool base-mount 785 that can be used in the accessibility system 700. The rotating multiple tool base-mount 785 rotates around so that a user can attach and detach to various objects. Vertical projections 830 are provided so that various objects attached to a corresponding universal tool mount 201 with universal tool mount cavity 250 can be placed on the rotary base-mount 785. The user simply can push the outside edge of the rotary base-mount to position the desired tool or object they want to latch onto. The rotary base-mount 785 can be placed on a surface such as a table or it can be connected to a clamp 880 with an adjustable arm 790 and a quick release 795 to be positioned directly above the lap of an individual who is either in bed or in a wheelchair. FIG. 34B shows a top view of the rotating multiple tool base-mount 785 and FIG. 34C shows a perspective view of the rotating multiple tool base-mount 785. As shown in FIGS. 34A-34C, the rotating base-mount is skeletonized with base-mount openings 815 so that tools connected to universal tool mounts 201 can extend below the rotary mount if necessary. A rotary knob 825 can be tightened or loosened to adjust to how easily it is to spin the rotary base-mount. The joint from the bottom of the rotating base-mount 785 to the clamp 880 has a quick release mechanism 795 so that a user or other person can quickly take the fixture off while the clamp 880 remains fixed on a surface. This is useful for individuals who may need to transfer in and out of bed or a wheelchair.

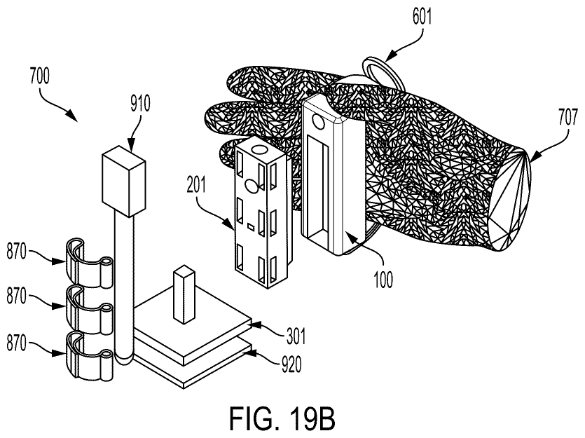

FIG. 19A is an exploded side view of the adapter 100 and single base-mount system 301 used for a toothbrush 910. FIG. 19B is an exploded perspective view of the adapter 100 and single base-mount system 301 used for a toothbrush 910. The accessibility system 700 of FIGS. 19A and 19B, may be used in a user's bathroom where the singular base mount 301 may be adhered with adhesive material 920, such as double-sided adhesive or loop-lock Velcro material, on a surface such as bathroom sink counter.

FIGS. 20A-20E, as described herein above, is an illustrative example of the multiple tool base-mount 800 adhered to a table-top 930 via double sided adhesive or Velcro 960 as shown and described with reference to FIG. 21B. This is a setup which may be used in a user's home on a dining table, bedside table, or a hospital bedside table, or other surfaces. Common tools are placed on the vertical projections 830, such as a fork 701 (as shown in FIG. 24), a spoon 940, a knife 951, and a cup 840. The fork 701 and spoon 940 can be adjusted due to being attached to the universal tool mount 201 via ball joint 702 and socket 703. A knife 951 consisting of the same magnetic and mechanical latching features as the universal tool mount 201 or cup 840 are shown on the user's side of the knife 951. There is also a circular blade, 952, which extends out of the bottom of the knife's body 951. The circular knife 952 can either be tightened to the knife's body 951 so that the blade may not rotate which allows the user to push down on food to cut. The circular blade 952 can also be loosened so that the blade can freely rotate in the knife's body so that the user may roll the knife body over food and slice through food similar to a pizza cutter.

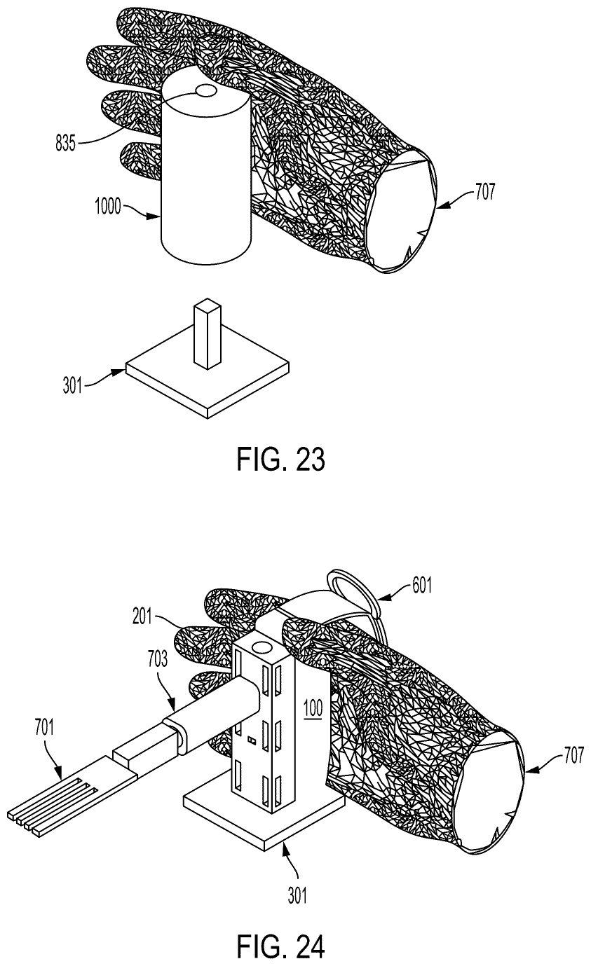

FIG. 22 illustrates the wide-grip tool 1000 nested onto the singular base-mount 301. The wide-grip tool is used for individuals with some hand (or other extremity) movement, hence having a wider cylindrical grip makes it easier to grip due to less finger flexion around the wide-grip tool compared to other objects such as conventional utensils. The wide-grip tool 1000 has the same rectangular cavity as the universal tool mount so that the wide-grip tool can be nested onto a singular base-mount when not in use. This allows the user with limited hand movement to have an easier reach to grasp movement than picking it up off a surface such as a table. FIG. 23 illustrates the wide-grip tool expanded when it is in use. At the top of the wide-grip tool 1000, there is a threaded hole 835, similar to the threaded hole 230 at the top of the universal tool mount 201. Threaded hole 835 allows a universal joint 702 and socket 703 (not shown) to be connected to the wide-grip tool 1000 so that different tools and utensils can be connected and articulated in various angles to accommodate the user's preferences.

In accordance with various aspects, the invention provides an adapter 100 including cavity on the palmar side for latching and a cavity behind said cavity 160 on the palmer side for a magnet. The cavity on the palmar side may be for latching has a recessed cavity for latching. The adapter 100 may have a threaded hole 130 on the palmar side for connecting tools or universal joints. In accordance with further aspects, the invention provides a singular base-mount including a vertically extending portion 310, and a threaded hole 330 on the bottom. In accordance with further aspects, the invention provides a universal tool mount 201 including a cavity 250 to nest onto a vertical protrusion 830 and a cavity 260 inside the rectangular extrusion for a magnet. The universal tool mount 201 may have a threaded hole 230 on the front face. The universal tool mount may also or alternately have a threaded hole 230 on the top face. The universal tool mount 201 may also or alternately have strap holes 210. The universal tool mount 201 may have a latch projection 270 as shown at the top to latch the adapter. In accordance with further aspects the invention provides tool extender apparatus 401 including a threaded hole 430, a universal joint (e.g., ball and socket joint) connected to said thread hole and strap holes 210.

### Spring Latch Mechanism

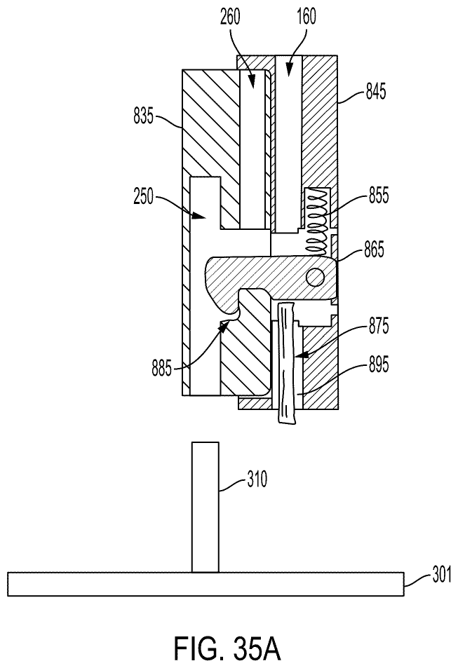

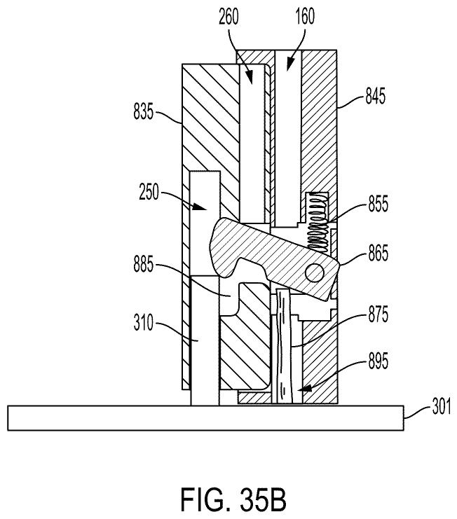

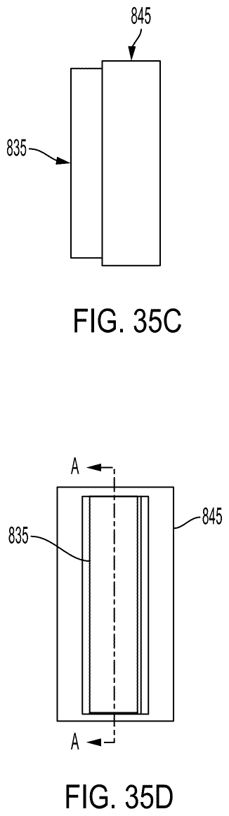

A first alternative aspect of the invention is provided with reference to FIGS. 35A-35D, an adapter 845 and universal tool mount 835 is shown with a spring latch mechanism to latch the adapter 845 to the universal tool mount 835 during use, as described herein above. FIG. 35A shows the adapter 845 and universal tool mount 835 in a side cross-sectional view.

The adapter 845 includes a latch 865 that is biased in position by a spring 855 and as shown in FIG. 35A, the latch 865 is engaged to the catch 885 on the universal tool mount 835. The magnet cavity 260 on the universal tool mount 835 may contain a magnet that attracts to a magnet in the magnet cavity 160 in the adapter 845. The single base mount 301 and vertically extending portion 310 is not engaged in the cavity 250 when the adapter 845 being latched to the universal tool mount 835 during use. FIG. 35B shows the adapter 845 engaged to the universal tool mount 835 held in place by the magnetic attraction of the magnet within the cavity 160 of the adapter 845 to the magnet within the cavity 260 of the universal tool mount 835, with the universal tool mount 845 positioned on the vertically extending portion 310 of the single base mount 301. In this position, the vertically extending portion 310 displaces the latch 865 against the bias of the spring 855 to release the latch 865 from the catch 885. The user can make a slight supination movement to disengage the adapter 845 from the universal tool mount 835. Within the adapter, the spring latch may consist of a magnet cavity for a magnet to be inserted to be attracted to a magnet within the connector. When the adapter 845, which is on a user's hand (or other extremity), comes in contact with the universal tool mount 835, the magnets pull the universal tool mount 835 and adapter 845 towards each other, allowing a gross movement for someone with arm impairments to easily align the universal connector and adapter. FIG. 35C shows a side view of the adapter 845 engaged with the universal tool mount 835 showing the portion of the universal tool mount 835 nested in the adapter 845. FIG. 35D further depicts the arrangement from a front view. As the user and magnets pull the adapter 845 towards the universal tool mount 835 the catch 885 pushes the latch 865 up, over, and around to be in the locked position as seen in FIG. 35A. The nested arrangement of the adapter 845 and the universal tool mount 835, as depicted in FIGS. 35C and 35D, allow for the universal tool mount 835 to be secured from up/down and side to side movements. The latch 865 to catch 885 arrangement secures the third dimension of movement, therefore providing a stable and secure connection in all three dimensions while the individual uses the corresponding tool.

Optionally, to release the adapter 845 from the universal tool mount 835, a rod 875 can be provided to release the latch 865 from the catch 885. As the rod 875 is inserted in the rod cavity 895, the rod 875 presses the latch 865 up against the bias of the spring 855, freeing the adapter 845 from the universal tool mount 835. The rod 875 can be permanently retained in the adapter 845 or inserted only when required for removal.

Alternatively, the rod 875 can be sized so that placement of the universal tool mount 835 on a vertically extending portion 310 (or vertical projection 830 on a base-mount 800) displaces the rod 875 to raise the latch 865 from the catch 885. This method requires the rod 875 to protrude out under the adapter 845 during use. If necessary, the user (or a caregiver) can press down on the adapter 845, hitting the protruding rod 875 and releasing the adapter 845 from the universal tool mount 835.

One skilled in the art will appreciate that the relative position of the elements of the illustrative embodiment described with reference to FIGS. 35A-35D can be altered without departing from the scope of the invention. For example, a spring 855 can be provided with a bias in compression or tension. Similarly, the latch 865 can reside on the universal tool mount 835 with a corresponding catch 885 on the adapter 845. The methods described herein are possible to perform to provide assistance to use ordinary objects with one arm and without any hand (or other extremity) mobility.

### Base-Mount and Rod

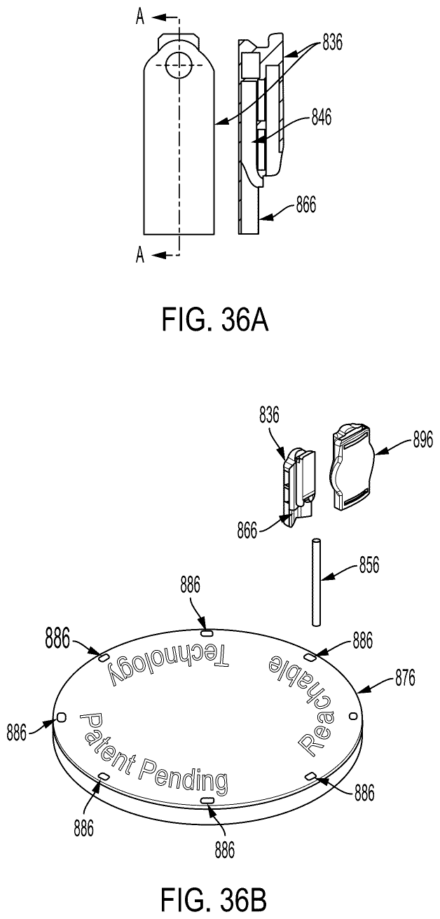

A second alternative aspect of the invention is provided with reference to FIGS. 36A and 36B. FIG. 36A shows a rear and side view of a universal tool mount 836 with cavities 846 to allow for one of a plurality of rods 856 to be inserted and easily removed based on user preference. FIG. 36B shows an exploded perspective view of the base-mount 876 with various base-mount holes 886 into which one of a plurality of rods 856 can be inserted, each of which can accommodate a universal tool mount 836. The one of a plurality of rods 856, each inserted into the base-mount holes 886 is used to store the universal tool mount 836 or other components with rod cavities 846 such as the cup holder, as described herein above. The base-mount holes 886 securely hold the rod 856 so that the universal connector can be slid onto the rod.

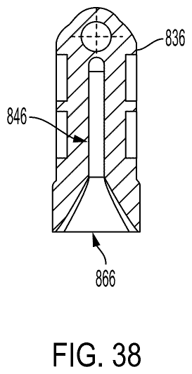

The universal tool mount 836 has a cavity 846 for the rod to be inserted inside. There is sufficient clearance within the rod cavity 846 so that the user can easily slide the universal tool mount 836 on and off of the rod 856 secured onto the base-mount 876. Once the universal tool mount 836 is engaged to the rod 856 with the rod 856 inside the cavity 846, the user can detach the adapter 896 from that specific universal tool mount 836, storing it on that specific rod 856 for later use. As depicted in FIGS. 36A and 36B, and as further depicted in FIG. 38, the cavity 846 has a tapered opening 866 such as a v shaped wall so that the user can use more gross movements and engage the rod 856 to the tapered opening 866 which makes it easier to nest into the cavity 846. FIG. 38 depicts cross-sectional view of the universal tool mount 836 showing the tapered opening 866 as a lead-in to the cavity 846.

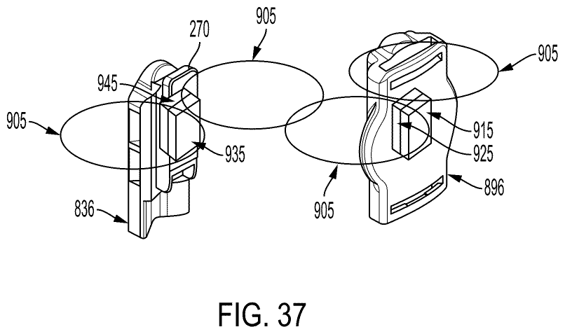

FIG. 37 depicts another aspect of the invention that provides assistance to users. FIG. 37 shows a perspective view of the universal tool mount 836 in proximity to the adapter 896 (with the strap 601 and user hand (or other extremity) 707 not shown for clarity). The mechanism for case of use for attachment of the adapter 896 to the universal tool mount 836 is the magnetic attraction from the magnet within the universal tool mount 836 and the magnet within the adapter 896. As described above, there is a latch protrusion 270 to secure the adapter and universal connector together. Additionally, the magnetic attraction not only helps secure the coupling of adapter 896 and universal tool mount 836, but the magnetism guides the coupling, allowing individuals with limited mobility to latch easily. As shown in FIG. 37, the universal tool mount 836 and adapter 896 both have cavities for a magnet. The polarity of the of the magnets are oppositely placed within the cavities such that the north side of the magnet within the adapter 915 is aligned with the south side of the magnet within the tool mount 945, and the south side of the magnet within the adapter 925 is aligned with the north side of the magnet within the tool mount 935. This creates a magnetic attraction between the universal tool mount 836 and the adapter 896. As the user brings the adapter 896 closer to the universal tool mount 836, the magnetic attraction seen in FIG. 37 with the overlapping magnetic fields 905 helps guide the user to easily latch the adapter onto the universal connector. In accordance with further aspects, one or the other of the adapter and the universal connection pair may include a magnet and the other of the pair may include a ferromagnetic material. This method of magnetic attraction to secure various parts allows for a secure connection with ease of use via magnetic attraction.

In accordance with certain aspects therefore, the invention provides a method of operating the adapter via magnetic attraction to guide movement for individuals with limited hand (or other extremity) mobility. By having a magnet in the adapter and the connector, the attraction snaps them together, which helps tremendously for people with limited hand mobility.

The base-mount 876 may include rods 856 that may be removable. As shown in FIG. 36B The latching function between the adapter 845 and the universal tool mount 835 may include a spring 855 and latch 865 mechanism which secures the universal connector with the adapter, or a latch mechanism that can be completed without the help of another hand. The entire latching procedure can be done without any fine hand movement. The latch can be decoupled by inserting the rod in the universal connector's rod cavity or by pressing down on the latch release bar. The latter option allows individuals to decouple without the base-mount allowing for more options.

Those skilled in the art will appreciate that numerous modifications and variations may be made to the above disclosed embodiments without departing from the spirit and scope of the present invention.

What is claimed is:

1. An accessibility system for assisting with access and use of any of a plurality of tools, the accessibility system comprising:
a base-mount for fixed attachment within an environment, the base-mount including at least one vertically extended portion;
an adapter for attachment to an extremity of a user without the need to grip the adapter; and
a universal connector including a tool attached thereto, and a base-mount cavity for receiving the vertically extending portion of the base-mount, the adaptor and the universal connector each include one of a latch projection and a latch cavity that receives the latch projection.

2. The accessibility system of claim 1, wherein the latch projection includes a projection on an end of the universal connector about which the adaptor may be pivotally attached to the universal connector.

3. The accessibility system of claim 1, wherein the latch projection includes a pivotable latch within the universal connector.

4. The accessibility system of claim 1, wherein the vertically extending projection disengages the latch projection from the latch cavity when the universal connector is positioned on the vertically extending portion.

5. The accessibility system of claim 1, wherein the base-mount further includes a plurality of vertically extended portion, each of the plurality of vertically extended portions including a magnetic insert to releasably secure the position of the corresponding one of a plurality of universal connectors relative to the extended projection when the latch projection is inserted into the latch cavity.

6. The accessibility system of claim 1, wherein at least one of the adaptor and the universal connector further includes a magnet for assisting in maintaining contact between the adaptor and the universal connector.

7. The accessibility system of claim 1, wherein at least one of the universal connector, the adaptor and the tool includes an extender with at least one ball-joint.

8. The accessibility system of claim 1, wherein the tool includes at least one of a kitchen utensil, a cup, a toothbrush, a phone holder, and a stylus.

9. An accessibility system for assisting with access and use of any of a plurality of tools, the accessibility system comprising:
a base-mount for fixed attachment within an environment, the base-mount including a plurality of vertically extended portions;
an adapter for attachment to an extremity of a user without the need to grip the adapter, the adapter including a latch cavity; and
a universal connector including a tool attached thereto, the adaptor and the universal connector including a latch projection and a latch cavity that receives the latch projection, and a base-mount cavity for receiving a vertically extending portion of the base-mount of the plurality of vertically extending portions.

10. The accessibility system of claim 9, wherein the latch projection includes a projection on an end of the universal connector about which the adaptor may be pivotally attached to the universal connector.

11. The accessibility system of claim 9, wherein each latch portion includes a pivotable latch within the universal connector.

12. The accessibility system of claim 9, wherein each vertically extending portion disengages the latch projection from the latch cavity when the universal connector is positioned on the vertically extending portion.

13. The accessibility system of claim 9, wherein the base-mount is provided on a rotary platform.

14. The accessibility system of claim 9, wherein at least one of the adaptor and the universal connector further includes a magnet for assisting in maintaining contact between the adaptor and the universal connector.

15. The accessibility system of claim 9, wherein at least one of the universal connector, the adaptor and the tool includes an extender.

16. The accessibility system of claim 9, wherein the tool includes at least one of a kitchen utensil, a cup, a toothbrush, a phone holder, and a stylus.

17. A method of assisting a user with access to and use of any of a plurality of tools, the method comprising:
moving an adaptor that is attached to an extremity of a user toward a universal connector, the universal connector including a tool attached thereto and being mounted on a vertically extended portion of a base-mount;
contacting the adaptor with the universal connector to attach the universal connector to the adaptor; and
elevating the universal connector with the adaptor upward off of the vertically extended portion of the base-mount.

18. The method of claim 17, wherein the method further includes returning the universal connection to the vertically extended portion of the base-mount by lowering the universal connector onto the vertically extended portion.

19. The method of claim 17, wherein the method further includes latching the universal connector to the adaptor.

20. The method of claim 17, wherein the base-mount includes a plurality of vertically extended portions, each of which includes a universal connector and a tool.
